(19) United States
(12) Patent Application Publication
(10) Pub. No.: US 2007/0043266 A1
(43) Pub. Date: Feb. 22, 2007

(54) Autostatic surgical separator

(76) Inventor: Oscar Laucirica Gari, Sant Feliu de Llobregat (ES)

Correspondence Address: JORDAN AND HAMBURG LLP, 122 EAST 42ND STREET, SUITE 4000, NEW YORK, NY, 10168, US

(21) Appl. No.: 11/349,421

(22) Filed: Feb. 6, 2006

(30) Foreign Application Priority Data
Aug. 5, 2003 (ES) P 2003 01876

(51) Int. Cl. A61B 1/32

(52) U.S. Cl. 600/228; 600/231

(57) ABSTRACT

The present invention has as its object an autostatic surgical separator for performing surgical operations on patients. The separator comprises a filed opening ring being of the open or closed type and exteriorly provided with a succession of arcuate notches, pulling valvae of different configurations being provided with respective arms having a smooth surface and any cross-section and being made up as one-piece members or as articulated arms, fixing devices being of a fixed or of a tiltable type with respect to the field opening ring and provided for fixing the valvae arms, a multiarticulated supporting arm being apt to maintain the ring in position with respect to the operating table, and an orientable cold light being provided with a multiarticulated supporting arm being apt to be fixed to the ring and allowing to properly position said light.

### OBJECT OF THE INVENTION

The present invention has as its object an autostatic surgical separator being apt to be applied in the surgical operations being preferentially performed on the abdomen, thorax, back, perineum and lumbar region of a patient.

### BACKGROUND OF THE INVENTION

In order to perform surgical operations on patients a surgical incision is normally carried out in the first place thus opening the operative field to the intra-abdominal or corresponding contents of the patient in order to thus thereupon be in a position to perform the corresponding surgical operation.

Autostatic or self-retaining surgical separators are used in order to en bloc fix and separate the lips of the surgical incision to thus allow performing the operation.

There are several patent documents disclosing the corresponding separator assemblies as well as several of their components and accessories, and from among said documents can be cited, among others, the patents U.S. Pat. No. 4,254,763 and US Appl. 2002-0026101 to Codman & Shurtleff Inc. relating to the separator or retractor assembly; U.S. Pat. No. 6,241,659 (corresponding EP Application no. 1 090 589) and U.S. Pat. No. 5,375,481 to Codman & Shurtleff Inc. relating to the fixing mechanism or device being provided to fix the valvae arms; U.S. Pat. No. 5,520,608 to Johnson & Johnson Professional relating to a valva; and a number of components and accessories being disclosed in patents U.S. Pat. No. 5,365,921; U.S. Pat. No. 5,356,100; U.S. Pat. No. 5,320,314; U.S. Pat. No. 5,320,444 and U.S. Pat. No. 4,467,791 also to Codman and having been applied for by several inventors.

The precited Codman & Shurtleff patents have mechanical limitations as regards the slotting of the valvae pulling arms.

Said drawbacks have been partially eliminated by the Spanish Utility Model no. 200102527 (Publ. no. ES 1 050 254 U) of this same applicant notably improving the fixing device being provided to fix the valvae arms as well as the very arms and the lighting of the patient's region.

Said utility model essentially comprises the field opening ring, pulling valvae of several dimensions and configurations being provided with pulling arms of square cross-section having their upper face slotted, fixing devices being each provided with a rectangular channel in correspondence with the valvae pulling arms thus allowing to fit and lock/unlock said arms in order to thus fix said valvae in position, and orientable lighting means.

The precited, already granted Spanish utility model has nevertheless some limitations as regards its operation and use, and it has a complex makeup as regards the valvae arms and the corresponding fixing devices.

### SUMMARY OF THE INVENTION

The above-mentioned and other drawbacks and limitations regarding the makeup of the previous utility model and of the different patents having been cited above, especially the Codman patents, have now been totally obviated by means of the surgical separator of the present invention.

The separator being the object of the present invention has notable differences as compared with the precited documents, and especially with the Codman patents, and from among its main features it can be pointed out that the valvae arms are in this case not slotted, the separator of the invention hence allowing to incorporate the valva in any position (this not being possible in Codman) and to “micrometrically” retract the valva (there are no limitations as defined by a slotting of the arm), the separator offers a better hygiene and a better comfort as regards the positions being occupied by the different members of the surgical team around the operative field, the external fixation device is much more versatile, more reduced and less heavy than in Codman and is apt to be much more rapidly fitted, and the lighting device being provided with a cold light provides an orientable lamp (not being provided by Codman). Codman also has in the slot of the base plate of the fixing device a curved projection such that when the fixing device is fitted to the ring said curved projection can only be fitted into the valley of the corresponding notch of the ring, whereas the fixing device of the instant invention is at the bottom of said slot advantageously devoid of such projection, so that the fixing device of the invention can be fitted at the valley or at the peaks of the ring notches and is thus better adaptable to the ring for its fixation, and the valva can also be better adapted to the tissue planes.

The valvae arms of the Codman patents have a quadrangular cross-section and can hence only fit in one position into the longitudinal channel having a corresponding cross-section in the respective fixing device, whereas in the present invention the valvae arms are made up with any cross-section, this latter being preferentially circular or oval-shaped as the case might be, in the preferential embodiment being illustrated in the drawings the valvae arms of the invention hence having a round cross-section in correspondence with the same cross-section of the longitudinal channel of the fixing device, this allowing the valvae arms of the invention to be fitted into the fixing device channel in any angular position.

One object of the instant invention consists in an autostatic surgical separator for automatically and en bloc holding and separating the skin and the tissue planes around the lips of the surgical incision, said autostatic surgical separator being of the type comprising field opening means being provided for opening the field within said lips, valvae means being provided for pulling said lips and fitted into respective fixing devices being applied along the opening means, supporting means being provided for supporting the opening means, and lighting means being provided for lighting the field area; said autostatic surgical separator being essentially characterised in that it comprises in combination:

field opening means being made up by way of ring having a closed and/or open configuration;

pulling valvae means of different configurations being integral with the corresponding pulling arm having a smooth surface and any cross-section, this latter being preferentially circular or oval-shaped;

fixing devices allowing to fix respective pulling valvae being fitted into them by means of the corresponding arms to the field opening ring, said fixing devices comprising a central body being fitted to a slotted base plate by means of which the fixing device is fixed to the field opening ring, said central body having a longitudinal channel whose cross-section is in correspondence with the cross-section of the pulling arm of the respective valva, said central body as well upperly having a transversal slit allowing to fit into it an apertured dog being made up by a plate being provided with an aperture whose contour is with a slight play in correspondence with the valva arm cross-section, said plate being provided with an upper appendage making up the dog itself, the valva pulling arm with a slight play sliding through said aperture of the dog plate, said upper apertured dog being permanently biased in a forward direction by a stub being in its turn biased by resilient means, said arrangement allowing the valva pulling arm to slide through the aperture of the plate of the upper apertured dog when sliding through the longitudinal channel, said aperture of the apertured dog locking the valva pulling arm when this latter attempts to move in the same direction and in the opposite sense, the pulling arm thus being locked inside the fixing device, the locking pressure being exerted by the apertured dog on the pulling arm becoming higher when there is an increase of the pressure being exerted on the valva, said locking being released when the user proceeds to digitally actuate said apertured dog in a backward direction thus allowing the corresponding valva pulling arm to be easily slid with respect to the dog aperture; said fixing device being of a fixed and/or tiltable type with respect to the field opening ring;

a multiarticulated supporting arm being provided to support the field opening ring and having a mechanism allowing to quickly and easily fix one end of said arm to the corresponding support and the opposite end to the field opening ring, said arm comprising two tubular structures being articulated at one of their ends by a central locking thread and at the other end of each tube having a ball joint arrangement at one side articulating the rod being incorporated in the lateral support for the arm and at the other side articulating the clamp being provided to fix the field opening ring; and

an orientable cold light being provided at the end of a multiarticulated arm being similar to the above-mentioned supporting arm and having a mechanism allowing to quickly and easily fix one end of said arm to the field opening ring, said orientable light being provided at the opposite end.

The field opening ring having an open or closed configuration is essentially planar and is exteriorly provided with a succession of arcuate notches.

The pulling arms have a smooth surface and any cross-section, this latter preferentially being circular and/or oval-shaped in correspondence with the longitudinal channel of the fixing device and the aperture of the apertured dog of the fixing device, said aperture and arm having the same cross-section with a slight play. Said pulling arms are each made up as a rigid, one-piece member, or else they can be articulated at an intermediary position thus occupying less space.

The fixing devices being provided to fix the valvae arms to the field opening ring are of a fixed and/or tiltable type with respect to said ring. The tiltable fixing device comprises a swivel pin being located at the front face between the central body and the slotted base plate and allowing to carry out an angular motion between both parts, said angular motion being lockable at different angular values by means of the incorporation of a tubular structure having an arcuate concave front face and establishing contact with a projection of the back face of the base plate of the fixing device, a special spring being provided which maintains the arcuate, tubular structure with the locking notches in position against the base plate body thereby maintaining the inclination of the fixing device main body and hence of the corresponding valva arm. The fixing device of the fixed type in its turn consists in a one-piece member integrally comprising the central body and the slotted base plate.

As for the multiarticulated supporting arm, a second tube is apt to slide inside each tube, said second tube ending in a 45° chamfering at the level of the central locking thread, in such a way that in said locking arrangement the locking thread articulates two bodies having a tapered section such that when threadingly tightening the assembly the taperingly driving bodies are brought nearer to one another thus simultaneously pushing the inner tubes of each tube against the ball joint arrangement being provided at each end, the whole assembly making up the articulated arm being thus totally locked, a three-dimensional movability being thus provided for the ball joint arrangement in combination with the thread.

The orientable cold light has a mechanism comprising two articulated tubular structures, said mechanism being similar to that of the multiarticulated arm having been described above. Said light comprises a lamp being made up by the precited multiarticulated support, a support being apt to be fitted to the field opening ring, a cup being provided with an optical lens at its end, the cup being threadingly engaged with the multiarm support and at the free end being provided with a thread for the fixation of the optical lens, said cup at the opposite end having a thread allowing to fit the corresponding optical fibre tube.

Another object of the present invention consists in the field opening ring which can be of the open or of the closed type, and the pulling arms pertaining to an autostatic surgical separator for automatically and en bloc holding and separating the skin and the tissue planes around the lips of the surgical incision, said autostatic surgical separator being of the type comprising field opening means for opening the field within said lips, valvae means being provided for pulling said lips and fitted into respective fixing devices being applied along the opening means, supporting means being provided for supporting the opening means, and lighting means being provided for lighting the field area; said autostatic surgical separator being essentially characterised in that the field opening ring of the open/closed type is essentially planar and is exteriorly provided with a succession of arcuate notches, and in that the pulling arms have a smooth surface and any cross-section, this latter preferentially being circular and/or oval-shaped, the aperture of the plate of the apertured dog of the fixing device in correspondence with said cross-sectional shape having the same cross-section with a slight play being preferentially existent in the vertical plane. The pulling arms are made up by a rigid one-piece member, or else they are articulated at an intermediary position and thus occupy less space.

Another object of the instant invention consists in the fixing devices being provided to fix the valvae arms to the field opening ring, said fixing devices being applicable to an autostatic surgical separator for automatically and en bloc holding and separating the skin and the tissue planes around the lips of the surgical incision, said autostatic surgical separator being of the type comprising field opening means for opening the field within said lips, valvae means being provided for pulling said lips and fitted into respective fixing devices being applied along the opening means, supporting means being provided for supporting the opening means, and lighting means being provided for lighting the field area; said autostatic surgical separator being essentially characterised in that the fixing devices being provided to fix the valvae arms to the field opening ring are of a fixed and/or tiltable type with respect to said ring. The tiltable fixing device comprises a swivel pin being located at the front face between the central body and the slotted base plate and allowing to carry out an angular motion between both parts, said angular motion being lockable at different angular values by means of the incorporation of a tubular structure having an arcuate concave front face and establishing contact with a projection of the back face of the base plate of the fixing device, a special spring being provided which maintains the arcuate, tubular structure with the locking notches in position against the base plate body. The fixing device of the fixed type in its turn consists in a one-piece member integrally comprising the central body and the slotted base plate.

Another object of the present invention consists in a multiarticulated supporting arm being provided to support the field opening ring, said multiarticulated supporting arm being applicable to an autostatic surgical separator for automatically and en bloc holding and separating the skin and the tissue planes around the lips of the surgical incision, said autostatic surgical separator being of the type comprising field opening means for opening the field within said lips, valvae means being provided for pulling said lips and fitted into respective fixing devices being applied along the opening means, supporting means being provided for supporting the opening means, and lighting means being provided for lighting the field area; said autostatic surgical separator being essentially characterised in that in the mechanism of the multiarticulated supporting arm a second tube is apt to slide inside each tube, said second tube ending in a 45° chamfering at the level of the central locking thread, in such a way that in said locking arrangement the locking thread articulates two bodies having a tapered section such that when threadingly tightening the assembly the taperingly driving bodies are brought nearer to one another thus simultaneously pushing the inner tubes of each tube against the ball joint arrangement being provided at each end, the whole assembly making up the articulated arm being thus totally locked, a three-dimensional movability being thus provided for the ball joint arrangement in combination with the thread.

Another object of the instant invention refers to the arrangement of an orientable cold light being applicable to an autostatic surgical separator for automatically and en bloc holding and separating the skin and the tissue planes around the lips of the surgical incision, said autostatic surgical separator being of the type comprising field opening means for opening the field within said lips, valvae means being provided for pulling said lips and fitted into respective fixing devices being applied along the opening means, supporting means being provided for supporting the opening means, and lighting means being provided for lighting the field area; said autostatic surgical separator being essentially characterised in that the cold light has a multiarticulated supporting arm mechanism wherein a second tube is apt to slide inside each tube, said second tube ending in a 45° chamfering at the level of the central locking thread, in such a way that in said locking arrangement the locking thread articulates two bodies having a tapered section such that when threadingly tightening the assembly the taperingly driving bodies are brought nearer to one another thus simultaneously pushing the inner tubes of each tubular member against the ball joint arrangement being provided at each end, the whole assembly making up the articulated arm being thus totally locked, a three-dimensional movability being thus provided for the ball joint arrangement in combination with the thread.

The orientable cold light consists in a lamp comprising the multiarticulated support, a support being provided to fit this latter to the ring, a cup and an optical lens, said cup being threadingly engaged with its support being provided at the multiarticulated arm, said cup at an end having a nut allowing to fix the optical lens and at the opposite end having a thread allowing to fit the corresponding optical fibre tube.

These and other features are being highlighted in the accompanying figures illustrating an exemplary embodiment of the invention having a merely illustrative character and in no way limiting the scope of the present invention.

### DESCRIPTION OF THE DRAWINGS

In said drawings:

FIG. 1 is a diagrammed, perspective view of the autostatic surgical separator.

FIG. 2 is a perspective view of the separator of the invention in use position on the patient.

FIG. 3 is a perspective view from the back showing the fixing device of the tiltable type being fitted to the ring.

FIG. 4 is an elevational view of the device of FIG. 3 having been in this case isolated.

FIG. 4A is a diagrammed elevation showing the device of FIGS. 3 and 4.

FIG. 5 is a perspective view from the back of the fixing device of the fixed type showing the valva arm being articulated at an intermediary position.

FIG. 6 is a diagrammed plan-view showing the set of components and accessories of the separator of the invention.

FIG. 7 is a plan-view of the cold light with the associated multiarticulated arm.

FIG. 8 is an exploded view of the multiarticulated supporting arm being provided to support the ring.

FIG. 9 is a view being similar to the FIG. 8 but in this case showing the clamping handle in its assembled state.

FIG. 10 is a diagrammed, fragmentary elevation showing the orientable cold light.

FIG. 11 is a diagrammed, essentially sectional, fragmentary view showing the gripping clamp being provided to allow the multiarticulated arm to grip the ring.

FIG. 12 is a diagrammed, perspective view of the gripper being provided to grip the supporting rod being provided to support the multiarticulated support, said gripper being provided with a clamp being apt to securely grip the support of the corresponding operating table.

### DETAILED DESCRIPTION OF A PREFERRED EMBODIMENT

According to the drawings the present invention consists in an autostatic surgical separator being generally referenced at —1— for automatically and en bloc holding and separating the skin and the tissue planes around the lips of the surgical incision, said autostatic surgical separator being of the type comprising field opening means being provided for opening the field within said lips, valvae means being provided for pulling said lips and fitted into respective fixing devices being applied along the opening means, supporting means being provided for supporting the opening means, and lighting means being provided for lighting the field area.

The present invention has as its object a separator —1— comprising in combination:

field opening means being made up by way of ring —2— having a closed and/or open configuration and any shape and size;

pulling valvae means —3— of different configurations being integral with the corresponding pulling arm —4— having a smooth surface and any cross-section, this latter being preferentially circular or oval-shaped, said pulling arm having been illustrated in the drawings with a circular surface, this latter being the preferred one;

fixing devices —5— allowing to fix respective pulling valvae —3— being fitted into them by means of the corresponding arms —4— to the field opening ring, said fixing devices comprising a central body —6— being fitted to a base plate —7— being slotted at —8— and by means of which the fixing device is fixed to the field opening ring —2—, said slot —8— being devoid of a projection of any type and thus having a flat bottom allowing the fixing device —5— to be fixed at the valleys or at the peaks of the notches of said ring —2—, said central body —6— of the fixing device —5— having a longitudinal channel —9— whose cross-section is in correspondence with that of the pulling arm —4— of the respective valva —3— (being illustrated in the drawings with a circular section, this being the preferred shape), said central body as well upperly having a transversal slit —10— allowing to fit into it an apertured dog —11— through which the arm —4— being provided for pulling the valva —3— is apt to slide, said upper apertured dog —11— being made up by a plate —11a— being provided with an aperture —11b— 1and an upper appendage —11c— acting as the very dog, said aperture —11b— having a contour that with a slight play is essentially in correspondence with the circular cross-section (or with the cross-section of any other shape) of the valva arm —4—, said dog —11— being permanently biased in a forward direction by a stub —12— being in its turn biased by resilient means being made up by a spring —13— and maintained in position by means of a screw —14—, said arrangement allowing the arm —4— being provided for pulling the valva —3— to slide through the aperture of the plate of the upper apertured dog —11— when sliding through the longitudinal channel —9— having been also illustrated with a circular cross-section (which is the preferential cross-section), said aperture —11b— of the plate —11a— of the apertured dog —11— locking the arm —4— being provided for pulling the valva —3— when said arm attempts to move in the same direction and in the opposite sense, the pulling arm —4— thus being locked inside the fixing device —5—, the locking pressure being exerted by the apertured dog —11— on the pulling arm —4— becoming higher when there is an increase of the pressure being exerted on the valva —3—, said locking being released when the user proceeds to digitally actuate said apertured dog —11— in a backward direction as per arrow —F— (FIG. 4A) thus allowing the corresponding arm —4— being provided for pulling the corresponding valva to be easily slid.

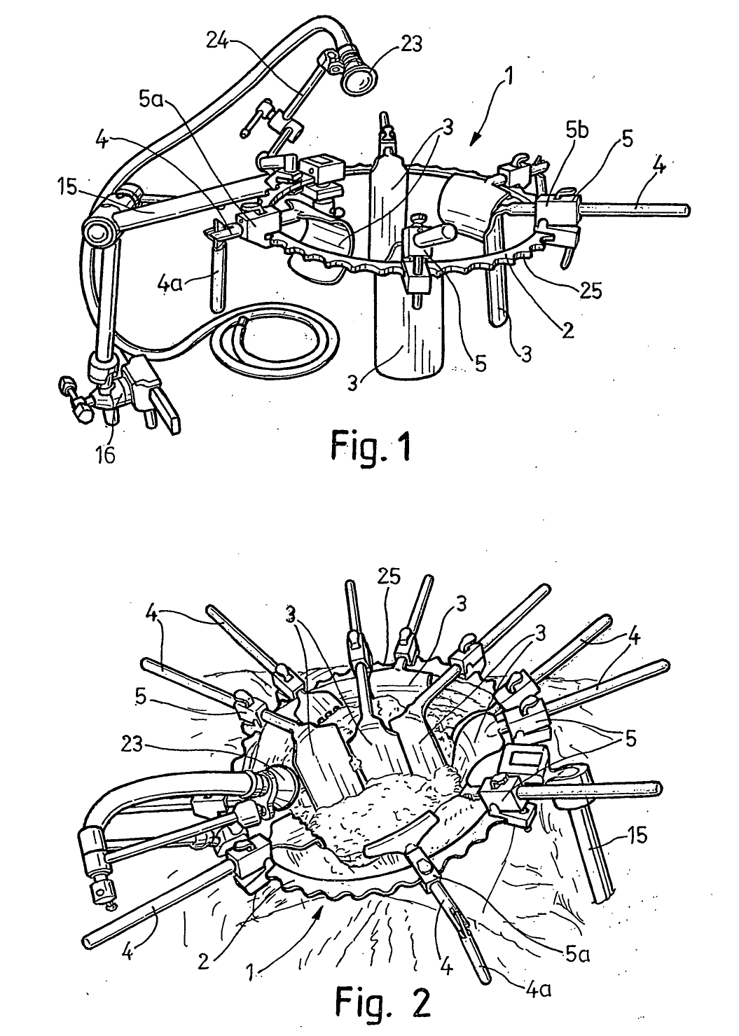

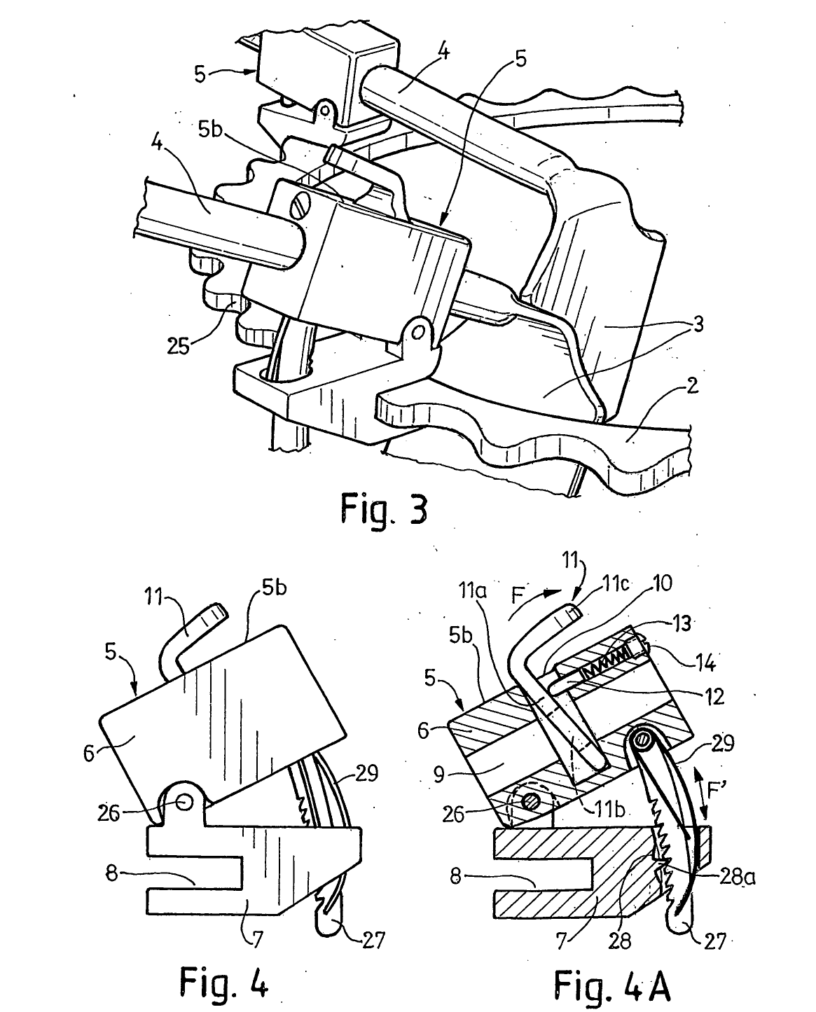

According to the invention the fixing device —5— can be of the fixed —5a— (FIGS. 1, 5 and 6) or of the tiltable —5b— type (FIGS. 1, 3, 4, 4A and 6) with respect to the field opening ring —2—.

The separator —1— comprises a multiarticulated supporting arm —15— (FIGS. 1, 2, 6, 8 and 9) being provided for supporting the field opening ring —2— and having a mechanism allowing to quickly and easily fix one end of said arm to the corresponding support —16— (FIGS. 1, 6 and 12) and the opposite end to the field opening ring —2— and comprising two tubular structures —17—, —18— being articulated at one —19— of their ends (FIGS. 8 and 9) by means of a central locking thread —20— and at the other end of each tube having a ball joint arrangement —21—, —22— at one side articulating the rod being incorporated into the lateral support being provided for supporting the arm, said ball joint arrangement at the other side articulating the gripping clamp being provided to fix the field opening ring.

The separator —1— comprises an orientable cold light —23— being provided at the end of a multiarticulated arm —24— being similar to the arm —15— of the above-mentioned support and having a mechanism allowing to quickly and easily fix one end of said arm to the field opening ring, said orientable light being provided at the opposite end.

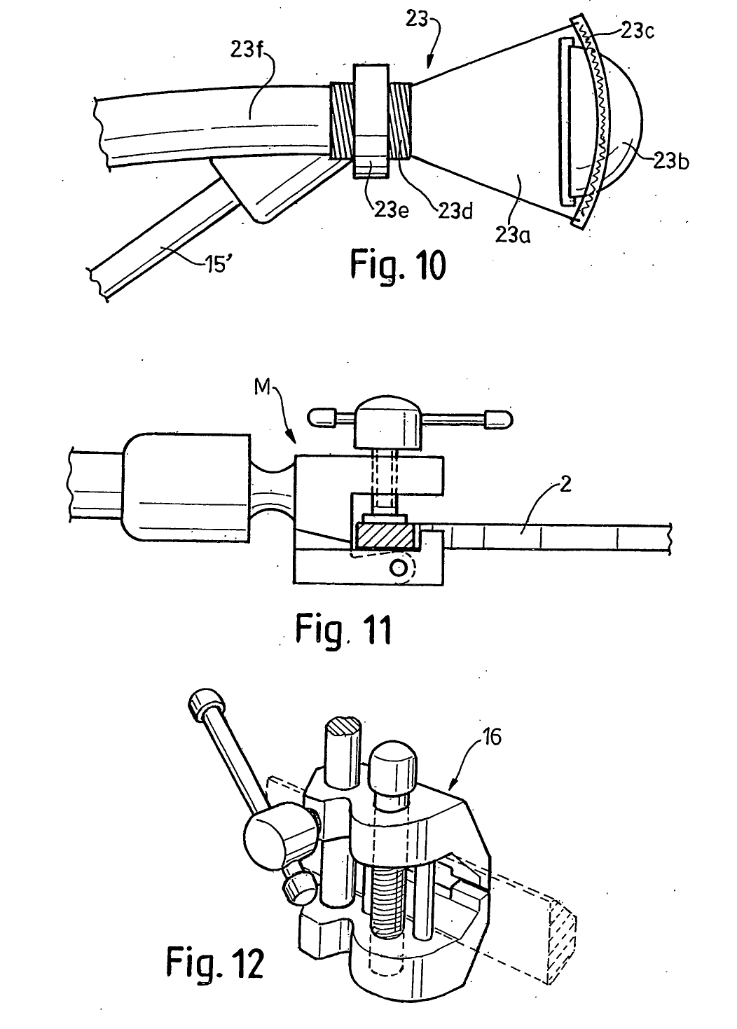

Said orientable cold light —23— comprises a hollow cup —23a— to one of whose ends an optical lens —23b— is fitted and for such a purpose is fixed by means of a nut —23c—, said cup being at the opposite end provided with a thread —23d— threadingly engaging the end —23e— of the support —15′—, this latter at its opposite end being fixed by means of the corresponding clamp to the ring —2—, said thread —23d— threadingly engaging the corresponding optical fibre cable —23f— (FIG. 10).

The field opening ring —2— being of the open or of the closed type is essentially planar and is exteriorly provided with a succession of arcuate notches —25—.

The pulling arms —4— have a smooth surface and any cross-section, this latter being preferentially circular (such as illustrated in the drawings) and/or oval-shaped, the aperture —11b— of the plate —11a— of the apertured dog —11— of the fixing device —5— in correspondence with said cross-sectional shape having the same cross-section with a slight play. Said pulling arms —4— are made up by a rigid one-piece member, or else they can be articulated —4a— at an intermediary position thus occupying less space.

The arms —4—, the longitudinal channel —9— of the fixing device —5— (—5a—, —5b—) and the aperture —11b— of the plate —11a— of the apertured dog —11— have been illustrated in the drawings with a circular cross-section or contour, this being the preferred configuration allowing said arms —4— to be fitted into the fixing device in any angular position.

The fixing devices —5— being provided for fixing the arms —4— of the valvae —3— to the field opening ring —2— are of the fixed —5a— and/or of the tiltable —5b— type with respect to said ring. The tiltable fixing device —5b— comprises a swivel pin —26— being located at the front face between the central body —6— and the slotted base plate —7— and allowing to carry out an angular motion between both parts as per the arrow —F′— (see FIG. 4A), said angular motion being lockable at different angular values by means of the incorporation of a tubular structure —27— having an arcuate concave front face and establishing contact with a projection —28a— of the back face —28— of the base plate —7— of the fixing device —5b—, a special spring —29— being provided which maintains the arcuate, tubular structure with the locking notches in position against the base plate body.

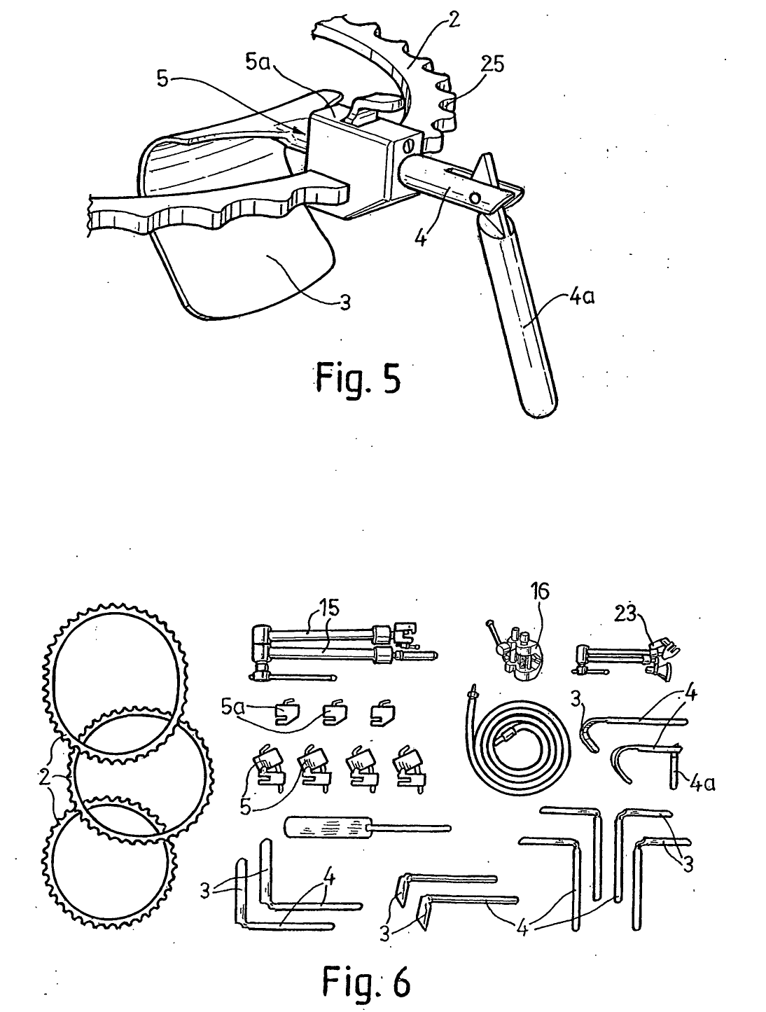

The fixing device of the fixed type —5a— in its turn consists in a one-piece member integrally comprising the central body and the slotted base plate as shown in FIG. 5.

As for the multiarticulated supporting arm —15—, a second tube —17′—, —18′— is apt to slide inside each tube —17—, —18—, said second tube ending in a 45° chamfering at the level of the central locking thread —20—, in such a way that in said locking arrangement the locking thread articulates two bodies having a tapered section such that when threadingly tightening the assembly the taperingly driving bodies are brought nearer to one another thus simultaneously pushing the inner tubes —17′—, —18′— of each tube against the ball joint arrangement being provided at each end, the whole assembly making up the articulated arm —15— being thus totally locked, a three-dimensional movability being thus provided for the ball joint arrangement in combination with the thread. Said multiarticulated supporting arm —15— is fixed to the ring by means of an articulated clamp arrangement —M— being apt to be tightened from the top by means of the corresponding thumbscrew or the like and to be adapted to any arc, thickness and size of the ring —2— in question (see FIG. 11).

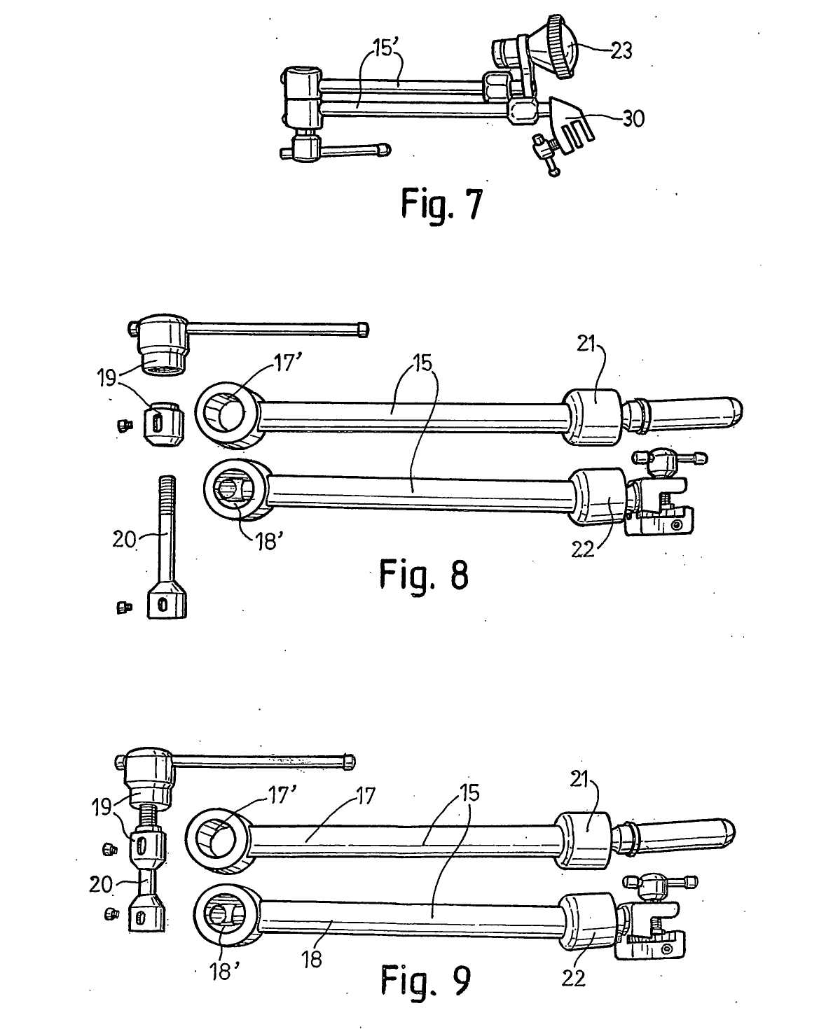

The orientable cold light —23— has a mechanism like that of the supporting arm —15— comprising two tubular structures —15′—, —15′— (FIG. 7) being articulated at one of their ends by means of a central locking thread and at the opposite end of each tube having a ball joint arrangement at one side articulating the rod being incorporated into the arm's lateral support being provided for supporting the lamp, said ball joint arrangement at the other side articulating the gripping clamp —30— being provided for the fixation to the field opening ring —2—, said mechanism being such that a second tube is apt to slide inside each tube, said second tube ending in a 45° chamfering at the level of the central locking thread, in such a way that in said locking arrangement the locking thread articulates two bodies having a tapered section such that when threadingly tightening the assembly the taperingly driving bodies are brought nearer to one another thus simultaneously pushing the inner tubes of each tubular member against the ball joint arrangement being provided at each end, the whole assembly making up the articulated arm being thus totally locked, a three-dimensional movability being thus provided for the ball joint arrangement in combination with the thread.

Another object of the present invention consists in the field opening ring —2— and the pulling arms —4— pertaining to an autostatic surgical separator —1— for automatically and en bloc holding and separating the skin and the tissue planes around the lips of the surgical incision, said autostatic surgical separator being of the type comprising field opening means for opening the field within said lips, valvae means being provided for pulling said lips and fitted into respective fixing devices being applied along the opening means, supporting means being provided for supporting the opening means, and lighting means being provided for lighting the field area.

Said object comprises the field opening ring —2— being essentially planar and exteriorly provided with a succession of arcuate notches —25—, and the pulling arms —4— having a smooth surface and any cross-section, this latter preferentially being circular and/or oval-shaped, the aperture of the apertured dog —11— of the fixing device —5— in correspondence with said cross-sectional shape having the same cross-section with a slight play being preferentially existent in the vertical plane. The pulling arms —4— can be made up by a rigid one-piece member, or else they can be articulated —4a— at an intermediary position so as to thus occupy less space and get as little as possible in the surgeon's way.

Another object of the instant invention consists in the fixing devices being provided to fix the valvae arms —4— to the field opening ring, said fixing devices being applicable to an autostatic surgical separator —1— for automatically and en bloc holding and separating the skin and the tissue planes around the lips of the surgical incision, said autostatic surgical separator being of the type comprising field opening means for opening the field within said lips, valvae means being provided for pulling said lips and fitted into respective fixing devices being applied along the opening means, supporting means being provided for supporting the opening means, and lighting means being provided for lighting the field area.

The fixing devices —5— being provided to fix the valvae arms —4— to the field opening ring —2— are of a fixed —5a— and/or tiltable —5b— type with respect to said ring —2—. The tiltable fixing device —5b— comprises a swivel pin —26— being located at the front face between the central body —6— and the slotted base plate —7— and allowing to carry out an angular motion (as per arrow —F′—, FIG. 4A) between both parts, said angular motion being lockable at different angular values by means of the incorporation of a tubular structure —27— having an arcuate concave front face and establishing contact with the back face —28— of the base plate —7— of the fixing device —5b—, a special spring —29— being provided which maintains the arcuate, tubular structure with the locking notches in position against the body of the base plate —7—.

The fixing device of the fixed type —5a— in its turn consists in a one-piece member integrally comprising the central body and the slotted base plate.

Another object of the present invention consists in a multiarticulated supporting arm —15— being provided to support the field opening ring —2—, said multiarticulated supporting arm being applicable to an autostatic surgical separator —i— for automatically and en bloc holding and separating the skin and the tissue planes around the lips of the surgical incision, said autostatic surgical separator being of the type comprising field opening means for opening the field within said lips, valvae means being provided for pulling said lips and fitted into respective fixing devices being applied along the opening means, supporting means being provided for supporting the opening means, and lighting means being provided for lighting the field area.

In the mechanism of the multiarticulated supporting arm —15— a second tube —17′—, —18′— is apt to slide inside each tube —17—, —18—, said second tube ending in a 45° chamfering at the level of the central locking thread —20—, in such a way that in said locking arrangement the locking thread articulates two bodies having a tapered section such that when threadingly tightening the assembly the taperingly driving bodies are brought nearer to one another thus simultaneously pushing the inner tubes —17′—, —18′— of each tube against the ball joint arrangement being provided at each end, the whole assembly making up the articulated arm being thus totally locked, a three-dimensional movability being thus provided for the ball joint arrangement in combination with the thread.

Another object of the instant invention refers to the arrangement of an orientable cold light being applicable to an autostatic surgical separator for automatically and en bloc holding and separating the skin and the tissue planes around the lips of the surgical incision, said autostatic surgical separator being of the type comprising field opening means for opening the field within said lips, valvae means being provided for pulling said lips and fitted into respective fixing devices being applied along the opening means, supporting means being provided for supporting the opening means, and lighting means being provided for lighting the field area.

The orientable cold light arrangement as per the invention has a mechanism of the multiarticulated supporting arm —24— wherein a second tube is apt to slide inside each tube —15′—, —15′—, said second tube ending in a 45° chamfering at the level of the central locking thread, in such a way that in said locking arrangement the locking thread articulates two bodies having a tapered section such that when threadingly tightening the assembly the taperingly driving bodies are brought nearer to one another thus simultaneously pushing the inner tubes of each tube against the ball joint arrangement being provided at each end, the whole assembly making up the articulated arm being thus totally locked, a three-dimensional movability being thus provided for the ball joint arrangement in combination with the thread.

According to the invention the separator as a whole and its different components and accessories are to be made of a stainless steel based material as befits the sanitary-hygienic maintenance having to be indispensably carried out for this type of instrumentarium.

It shall be understood that when practically embodying the autostatic surgical separator and the different components and accessories of the invention all detail changes as deemed opportune will be acceptable as long as they fall within the scope and spirit of the following claims.

1. Autostatic surgical separator for automatically and en bloc holding and separating the skin and the tissue planes around the lips of the surgical incision, said autostatic surgical separator comprising field opening means being provided for opening the field within said lips, valvae means being provided for pulling said lips and fitted into respective fixing devices being applied along the opening means, supporting means being provided for supporting the opening means, and lighting means being provided for lighting the field area;
said autostatic surgical separator further comprising in combination:
field opening means being made up by way of open/closed ring (2) of any configuration;
pulling valvae means (3) of different configurations being integral with the corresponding pulling arm (4) having a smooth surface and any cross-section, this latter being preferentially circular or oval-shaped;
fixing devices (5) allowing to fix respective pulling valvae (3) being fitted into them by means of the corresponding arms (4) to the field opening ring, said fixing devices comprising a central body (6) being fitted to a base plate (7) being provided with a slot (8) and by means of which the fixing device is fixed to the field opening ring (2), said central body having a longitudinal channel (9) whose cross-section is in correspondence with that of the pulling arm (4) of the respective valva (3), said central body as well upperly having a transversal slit (10) allowing to fit into it an apertured dog (11) being made up by a plate (11a) being provided with an aperture (11b) whose contour is with a slight play in correspondence with the cross-section of the valva arm (4), said plate being provided with an appendage (11c) making up the dog itself, the valva pulling arm (4) having a cross-section being with a slight play in correspondence with that of said aperture being apt to slide through said aperture (11b) of the dog plate, said upper apertured dog being permanently biased in a forward direction by a stub being in its turn biased by resilient means, said arrangement allowing the pulling arm (4) of the valva (3) to slide through the aperture (11b) of the plate of the upper apertured dog (11) when sliding through the longitudinal channel (9), the aperture of said apertured dog (11) locking the pulling arm (4) of the valva (3) when this latter attempts to move in the same direction and in the opposite sense, the pulling arm (4) thus being locked inside the fixing device (5), the locking pressure being exerted by the apertured dog (11) on the pulling arm (4) becoming higher when there is an increase of the pressure being exerted on the valva (3), said locking being released when the user proceeds to digitally actuate said apertured dog (11) in a backward direction thus allowing the pulling arm (4) of the corresponding valva (3) to be easily slid; said fixing device (5) being o fixed and/or tiltable tpe with respect to the field opening ring (2);
a multiarticulated supporting arm (15) being provided to support the field opening ring (2) and having a mechanism allowing to quickly and easily fix one end of said arm to the corresponding support (16) and the opposite end to the field opening ring (2) by means of an articulated, two-membered clamp arrangement (M), said arm comprising two tubular structures (15, 17, 18) being articulated at one of their ends by a central locking thread (20) and at the other end (21, 22) of each tube having a ball joint arrangement at one side (21) articulating the rod being incorporated in the lateral support for the arm and at the other side (22) articulating the clamp (M) being provided to fix the field opening ring (2); and
an orientable cold light (23) being provided at the end of a multiarticulated arm (15) being similar to the above-mentioned supporting arm and having a mechanism allowing to quickly and easily fix one end of said arm to the field opening ring, said orientable light being provided at the opposite end, said orientable cold light (23) comprising a cup (23a) being provided with a lens (23b) being fitted to one end and a thread (23d) at the opposite end, the multiarticulated support (15′) being fixed to said thread, the end of an optical fibre cable (23f) being fitted to said opposite end.

2. Autostatic surgical separator as per claim 1, wherein the field opening ring (2) having an open/closed configuration is essentially planar and is exteriorly provided with a succession of arcuate notches (25).

3. Autostatic surgical separator as per claim 1, wherein the pulling arms (4) have a smooth surface and any cross-section, this latter preferentially being circular and/or oval-shaped, the aperture of the plate of the apertured dog (11) of the fixing device (5) in correspondence with said cross-sectional shape having the same cross-section with a slight play being preferentially existent in the vertical plane.

4. Autostatic surgical separator as per claim 3, wherein the pulling arms (4) are made up by a rigid one-piece member.

5. Autostatic surgical separator as per claim 3, wherien the pulling arms (4) are articulated (4a) at an intermediary position thus occupying less space.

6. Autostatic surgical separator as per claim 1, wherein the fixing devices (5) being provided to fix the valvae arms (4) to the field opening ring (2) are fixed (5a) and/or tiltable (5b) with respect to said ring.

7. Autostatic surgical separator as per claim 6, wherein the tiltable fixing device (5b) comprises a swivel pin (26) being located at the front face between the central body (6) and the slotted base plate (7) and allowing to carry out an angular motion between both parts, said angular motion being lockable at different angular values by means of the incorporation of a tubular structure (27) having an arcuate concave front face and establishing contact with the back face (28) of the base plate (7) of the fixing device (5), a special spring (29) being provided which maintains the arcuate, tubular structure (27) with the locking notches in engagement with a projection (28a) of the body (28) of the base plate (7) thereby maintaining the fixing device (5b) in position with the desired inclination.

8. Autostatic surgical separator as per claim 6, wherein the fixed fixing device (5a) in its turn consists in a one-piece member integrally comprising the central body and the slotted base plate.

9. Autostatic surgical separator as per claim 1, wherein in the mechanism of the multiarticulated supporting arm (15) a second tube is apt to slide inside each tube, said second tube ending in a 45° chamfering at the level of the central locking thread, in such a way that in said locking arrangement the locking thread articulates two bodies having a tapered section such that when threadingly tightening the assembly the taperingly driving bodies are brought nearer to one another thus simultaneously pushing the inner tubes (17′, 18′) of each tubular member against the ball joint arrangement being provided at each end, the whole assembly making up the articulated arm being thus totally locked, a three-dimensional movability being thus provided for the ball joint arrangement in combination with the thread.

10. Autostatic surgical separator as per claim 1, wherein the mechanism of the orientable cold light (23) comprises two tubular structures (15′, 15′) being articulated at one of their ends by means of a central locking thread and at the opposite end of each tube having a ball joint arrangement at one side articulating the rod being incorporated into the arm's lateral support being provided for supporting the lamp, said ball joint arrangement at the other side articulating the gripping clamp being provided for the fixation to the field opening ring, said mechanism being such that a second tube is apt to slide inside each tube, said second tube ending in a 45° chamfering at the level of the central locking thread, in such a way that in said locking arrangement the locking thread articulates two bodies having a tapered section such that when threadingly tightening the assembly the taperingly driving bodies are brought nearer to one another thus simultaneously pushing the inner tubes of each tube against the ball joint arrangement being provided at each end, the whole assembly making up the articulated arm being thus totally locked, a three-dimensional movability being thus provided for the ball joint arrangement in combination with the thread.

11. Autostatic surgical separator as per claim 10, wherein the orientable cold light (23) consists in a lamp having a cup (23a) being provided at one end with an optical lens (23b) being held by means of a thread (23c), said cup at the opposite end having a thread (23d) where the multiarticulated support (15′) is fixed (23e), said multiarticulated support being at its other end fitted to the ring (2), the optical fibre tube (23f) being fitted to said thread.

12. Autostatic surgical separator as per claim 1, wherein the valvae arms (4), the aperture (11b) of the plate of the apertured dog (11) and the longitudinal channel (9) of the fixing device (5) preferentially have a circular cross-section/contour allowing to fit the arms into the fixing device in any angular position.

13. Autostatic surgical separator as per claim 1, wherein the bottom of the slot (8) of the base plate (7) of the fixing device (5) is flat and hence allows to fit and connect the fixing device (5) at the valleys and/or peaks of the notches (25) of the ring (2).

14. Autostatic surgical separator for automatically and en bloc holding and separating the skin and the tissue planes around the lips of the surgical incision, said autostatic surgical separator comprising field opening means being provided for opening the field within said lips, valvae means being provided for pulling said lips and fitted into respective fixing devices being applied along the opening means, supporting means being provided for supporting the opening means, and lighting means being provided for lighting the field area;
wherein the field opening ring (2) is essentially planar and exteriorly provided with a succession of arcuate notches (25), and in that the pulling arms (4) have a smooth surface and any cross-section, this latter preferentially being circular and/or oval-shaped, the aperture of the plate of the apertured dog (11) of the fixing device (5) in correspondence with said cross-sectional shape having the same cross-section with a slight play being preferentially existent in the vertical plane.

15. Autostatic surgical separator as per claim 14, wherein the pulling arms (4) are made up by a rigid one-piece member.

16. Autostatic surgical separator as per claim 14, wherein the pulling arms (4) are articulated (4a) at an intermediary position thus occupying less space.

17. Autostatic surgical separator as per claim 14, wherein the valvae arms (4), the aperture (11b) of the plate of the apertured dog (11) and the longitudinal channel (9) of the fixing device (5) preferentially have a circular cross-section/contour allowing to fit the arms into the fixing device in any angular position.

18. Autostatic surgical separator as per claim 14, wherein the bottom of the slot (8) of the base plate (7) of the fixing device (5) is flat and hence allows to fit and connect the fixing device (5) at the valleys and/or peaks of the notches (25) of the ring (2).

19. Autostatic surgical separator for automatically and en bloc holding and separating the skin and the tissue planes around the lips of the surgical incision, said autostatic surgical separator comprising field opening means being provided for opening the field within said lips, valvae means being provided for pulling said lips and fitted into respective fixing devices being applied along the opening means, supporting means being provided for supporting the opening means, and lighting means being provided for lighting the field area;
wherein the fixing devices (5) being provided for fixing the valvae arms to the field opening ring are ixed (5a) and/or tiltable (5b) with respect to said ring (2).

20. Autostatic surgical separator as per claim 19, wherein the tiltable fixing device (5b) comprises a swivel pin (26) being located at the front face between the central body (6) and the base plate (7) being provided with the slot (8), said swivel pin allowing to carry out an angular motion between both parts, said angular motion being lockable at different angular values by means of the incorporation of a tubular structure (27) having an arcuate concave front face and establishing contact with the back face of the base plate (7) of the fixing device (5b), a special spring (29) being provided which maintains the arcuate, tubular structure (27) with the locking notches in engagement with the body (28) of the base plate (7) in order to thus maintain the desired inclination.

21. Autostatic surgical separator as per claim 19, wherein the fixed fixing device (5a) consists in a one-piece member integrally comprising the central body and the slotted base plate.

22. Autostatic surgical separator as per claim 19, wherein the valvae arms (4), the aperture (11b) of the plate of the apertured dog (11) and the longitudinal channel (9) of the fixing device (5) preferentially have a circular cross-section/contour allowing to fit the arms into the fixing device in any angular position.

23. Autostatic surgical separator as per claim 19, wherein the bottom of the slot (8) of the base plate (7) of the fixing device (5) is flat and hence allows to fit and connect the fixing device (5) at the valleys and/or peaks of the notches (25) of the ring (2).

24. Autostatic surgical separator for automatically and en bloc holding and separating the skin and the tissue planes around the lips of the surgical incision, said autostatic surgical separator comprising field opening means being provided for opening the field within said lips, valvae means being provided for pulling said lips and fitted into respective fixing devices being applied along the opening means, supporting means being provided for supporting the opening means, and lighting means being provided for lighting the field area;
wherein in the mechanism of the multiarticulated supporting arm (15) a second tube (17′, 18′) is apt to slide inside each tube (17, 18), said second tube ending in a 45° chamfering at the level of the central locking thread, in such a way that in said locking arrangement the locking thread (20) articulates two bodies having a tapered section such that when threadingly tightening the assembly the taperingly driving bodies are brought nearer to one another thus simultaneously pushing the inner tubes of each tubular member against the ball joint arrangement being provided at each end, the whole assembly making up the articulated arm being thus totally locked, a three-dimensional movability being thus provided for the ball joint arrangement in combination with the thread, the ring (2) being gripped by means of an articulated gripping clamp (M).

25. Autostatic surgical separator for automatically and en bloc holding and separating the skin and the tissue planes around the lips of the surgical incision, said autostatic surgical separator comprising field opening means being provided for opening the field within said lips, valvae means being provided for pulling said lips and fitted into respective fixing devices being applied along the opening means, supporting means being provided for supporting the opening means, and lighting means being provided for lighting the field area;
wherein the orientable cold light (23) has a mechanism of the multiarticulated supporting arm (15) wherein a second tube is apt to slide inside each tube, said second tube ending in a 45° chamfering at the level of the central locking thread, in such a way that in said locking arrangement the locking thread articulates two bodies having a tapered section such that when threadingly tightening the assembly the taperingly driving bodies are brought nearer to one another thus simultaneously pushing the inner tubes of each tubular member against the ball joint arrangement being provided at each end, the whole assembly making up the articulated arm being thus totally locked, a three-dimensional movability being thus provided for the ball joint arrangement in combination with the thread.

26. Autostatic surgical separator as per claim 25, wherein the orientable cold light (23) consists in a lamp having a cup (23a) at one end incorporating an optical lens (23b) being held by means of a nut (23c), said cup at the opposite end having a thread (23d) where the corresponding end (23e) of the multiarticulated support (15′) is fitted, the end of an optical tube (23f) being also fitted to said thread.
